(19) United States
(12) Patent Application Publication
(10) Pub. No.: US 2007/0049641 A1
(43) Pub. Date: Mar. 1, 2007

(54) Methods for treating and monitoring inflammation and redox imbalance cystic fibrosis

(76) Inventors: Rabindra Tirouvanziam, Redwood City, CA (US); Lenore A. Herzenberg, Stanford, CA (US); Leonard A. Herzenberg, Stanford, CA (US); Carol Conrad, Menlo Park, CA (US); Richard B. Moss, Woodside, CA (US)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 11/507,706

(22) Filed: Aug. 22, 2006

Related U.S. Application Data
(60) Provisional application No. 60/710,807, filed on Aug. 24, 2005.

(51) Int. Cl. A61K 31/198

(52) U.S. Cl. 514/562

(57) ABSTRACT

The present invention relates to pharmaceutical kits and methods to treat lung inflammation and redox imbalance in human cystic fibrosis patients using pharmaceutical compositions containing N-acetylcysteine (NAC), pharmaceutically acceptable salts of N-acetylcysteine, or N-acetylcysteine derivatives. In phase I studies, treatment with oral NAC at a dose of from about 1800 mg/day to about 3000 mg/day for a period of 4 weeks produced significant positive effects, namely, it decreased absolute numbers of white blood cells and neutrophils in the sputum and produced concomitant decreases in sputum neutrophil elastase specific activity and sputum interleukin-8 levels, suggesting an amelioration of lung inflammation in the patients. These effects were associated with an increased total GSH level in whole blood as well increased staining for reduced GSH in blood neutrophils, both of which reflect an amelioration of the redox imbalance in the patients. In ongoing phase II studies, oral NAC at a dose of about 2700 mg/day administered in double-blind manner for 12 weeks showed excellent safety and significantly decreased white blood cells in sputum as compared to placebo.

### CROSS-REFERENCE TO RELATED APPLICATIONS

This application claims the benefit of priority to U.S. Provisional Application No. 60/710,807 (filed Aug. 24, 2005) entitled “Methods For Treating And Monitoring Inflammation And Redox Imbalance In Cystic Fibrosis,” the entire contents of which are incorporated by reference.

### FIELD OF THE INVENTION

The present invention relates to pharmaceutical kits and methods for treating lung inflammation and redox imbalance conditions in cystic fibrosis using pharmaceutical compositions comprising N-acetylcysteine, pharmaceutically acceptable salts of N-acetylcysteine, or pharmaceutically acceptable derivatives of N-acetylcysteine and a pharmaceutically acceptable carrier.

### BACKGROUND OF THE INVENTION

A free radical is a highly reactive and usually short-lived molecular fragment with one or more unpaired electrons. Free radicals are highly chemically reactive molecules. Because a free radical needs to extract a second electron from a neighboring molecule to pair its single electron, it often reacts with other molecules, which initiates the formation of many more free radical species in a self-propagating chain reaction. This ability to be self-propagating makes free radicals highly toxic to living organisms.

Living systems under normal conditions produce the vast majority of free radicals and free radical intermediates. They handle free radicals formed by the breakdown of compounds through the process of metabolism. Most reactive oxygen species come from endogenous sources as by-products of normal and essential metabolic reactions, such as energy generation from mitochondria or the detoxification reactions involving the liver cytochrome P-450 enzyme system. The major sources of free radicals, such as O₂⁻ and HNO₂⁻, are modest leakages from the electron transport chains of mitochondria, chloroplasts, and endoplasmic reticulum.

Reactive oxygen species (“ROS”), such as free radicals and peroxides, represent a class of molecules that are derived from the metabolism of oxygen and exist inherently in all aerobic organisms. The term “oxygen radicals” as used herein refers to any oxygen species that carries an unpaired electron (except free oxygen). The transfer of electrons to oxygen also can lead to the production of toxic free radical species. The best documented of these is the superoxide radical. Oxygen radicals, such as the hydroxyl radical (OH⁻) and the superoxide ion (O₂⁻) are very powerful oxidizing agents and cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, can be converted by biological systems into other more reactive species, such as peroxyl (ROO—), alkoxyl (RO—) and hydroxyl (OH—) radicals.

The major cellular sources of free radicals under normal physiological conditions are the mitochondria and inflammatory cells, such as granulocytes, macrophages, and some T-lymphocytes, which produce active species of oxygen via the nicotinamide adenine nucleotide oxidase (NADPH oxidase) system, as part of the body's defense against bacterial, fungal or viral infections.

Oxidative injury can lead to widespread biochemical damage within the cell. The molecular mechanisms responsible for this damage are complex. For example, free radicals can damage intracellular macromolecules, such as nucleic acids (e.g., DNA and RNA), proteins, and lipids. Free radical damage to cellular proteins can lead to loss of enzymatic function and cell death. Free radical damage to DNA can cause problems in replication or transcription, leading to cell death or uncontrolled cell growth. Free radical damage to cell membrane lipids can cause the damaged membranes to lose their ability to transport oxygen, nutrients or water to cells.

Biological systems protect themselves against the damaging effects of activated species by several means. These include free radical scavengers and chain reaction terminators; “solid-state” defenses, and enzymes, such as superoxide dismutase, catalase, and the glutathione peroxidase system.

Free radical scavengers/chemical antioxidants, such as vitamin C and vitamin E, counteract and minimize free radical damage by donating or providing unpaired electrons to a free radical and converting it to a nonradical form. Such reducing compounds can terminate radical chain reactions and reduce hydroperoxides and epoxides to less reactive derivatives.

The term “sold state defense” as used herein refers to the mechanism whereby a macromolecule binds a radical-generating compound, de-excites an excited state species, or quenches a free radical. The most important solid-state defense in the body is the black pigment melanin, which scavenges odd electrons to form stable radical species, thus terminating radical chain reactions.

Enzymatic defenses against active free radical species include superoxide dismutase, catalases, and the glutathione reductase/peroxidase system. Superoxide dismutase (SOD) is an enzyme that destroys superoxide radicals. Catalase, a heme-based enzyme which catalyses the breakdown of hydrogen peroxide into oxygen and water, is found in all living cells, especially in the peroxisomes, which, in animal cells, are involved in the oxidation of fatty acids and the synthesis of cholesterol and bile acids. Hydrogen peroxide is a byproduct of fatty acid oxidation and is produced by white blood cells to kill bacteria.

Glutathione, a tripeptide composed of glycine, glutamic acid, and cysteine that contains a nucleophilic thiol group, is widely distributed in animal and plant tissues. It exists in both the reduced thiol form (GSH) and the oxidized disulfide form (GSSG). In its reduced GSH form, glutathione acts as a substrate for the enzymes GSH-S-transferase and GSH peroxidase, both of which catalyze reactions for the detoxification of xenobiotic compounds, and for the antioxidation of reactive oxygen species and other free radicals. The term “xenobiotic” is used herein to refer to a chemical that is not a natural component of the organism exposed to it. Examples of xenobiotics include, but are not limited to, carcinogens, toxins and drugs. The metabolism of xenobiotics usually involves two distinct stages. Phase I metabolism involves an initial oxidation, reduction or dealkylation of the xenobiotic by microsomal cytochrome P450 monooxygenases (Guengerich, F. P. Chem. Res. Toxicol. 4: 391-407 (1991)); this step often is needed to provide hydroxyl- or amino groups, which are essential for phase II reactions. Glutathione detoxifies many highly reactive intermediates produced by cytochrome P450 enzymes in phase I metabolism. Without adequate GSH, the reactive toxic metabolites produced by cytochrome P-450 enzymes may accumulate causing organ damage.

Phase II metabolism generally adds hydrophilic moieties, thereby making a toxin more water soluble and less biologically active. Frequently involved phase II conjugation reactions are catalyzed by glutathione S-transferases (Beckett, G. J. & Hayes, J. D., Adv. Clin. Chem. 30: 281-380 (1993)), sulfotransferases (Falany, C N, Trends Pharmacol. Sci. 12: 255-59 (1991)), and UDP-glucuronyl-transferases (Bock, K W, Crit. Rev. Biochem. Mol. Biol. 26: 129-50 (1991)). Glutathione S-transferases catalyze the addition of aliphatic, aromatic, or heterocyclic radicals as well as epoxides and arene oxides to glutathione. These glutathione conjugates then are cleaved to cysteine derivatives primarily by renal enzymes and then acetylated, thus forming N-acetylcysteine derivatives. Examples of compounds transformed to reactive intermediates and then bound to GSH include, but are not limited to, bromobenzene, chloroform, and acetaminophen. Such toxicants may deplete GSH.

Depletion of GSH can diminish the body's ability to defend against lipid peroxidation. Glutathione peroxidase (GPx), an enzyme of the oxidoreductase class, catalyzes the detoxifying reduction of hydrogen peroxide and organic peroxides via oxidation of glutathione. GSH is oxidized to the disulfide linked dimer (GSSG), which is actively pumped out of cells and becomes largely unavailable for reconversion to reduced glutathione. GSH also is a cofactor for glutathione peroxidase. Thus, unless glutathione is resynthesized through other pathways, utilization of oxidized glutathione is associated with a reduction in the amount of glutathione available.

Glutathione reductase (NADPH), a flavoprotein enzyme of the oxidoreductase class, is essential for the maintenance of cellular glutathione in its reduced form (Carlberg & Mannervick, J. Biol. Chem. 250: 5475-80 (1975)). It catalyzes the reduction of oxidized glutathione (GSSG) to reduced glutathione (GSH) in the presence of NADPH and maintains a high intracellular GSH/GSSG ratio of about 500:1 in red blood cells.

Synthesis of GSH requires cysteine, a conditionally essential amino acid that must be obtained from dietary sources or by conversion of dietary methionine via the cystathionase pathway. If the supply of cysteine is adequate, normal GSH levels are maintained. But GSH depletion occurs if supplies of cysteine are inadequate to maintain GSH homeostasis in the face of increased GSH consumption. Acute GSH depletion causes severe—often fatal—oxidative and/or alkylation injury, and chronic or slow arising GSH deficiency due to administration of GSH-depleting drugs, such as acetaminophen, or to diseases and conditions that deplete GSH, can be similarly debilitating.

Cysteine is necessary to replenish hepatocellular GSH. Although various forms of cysteine and its precursors have been used as nutritional and therapeutic sources of cysteine, N-acetylcysteine (NAC) is the most widely used and extensively studied. NAC is about 10 times more stable than cysteine and much more soluble than the stable cysteine disulfide, cystine. Glutathione, glutathione monoethyl ester, and L-2-oxothiazolidine-4-carboxylate (procysteine/OTC) also have been used effectively in some studies. In addition, dietary methionine and S-adenosylmethionine are an effective source of cysteine.

Besides NAC's scavenger function, it is well-known that NAC promotes cellular glutathione production, and thus reduces, or even prevents, oxidant mediated damage. Indeed, treatment with NAC provides beneficial effects in a number of respiratory, cardiovascular, endocrine, infectious, and other disease settings as described in WO05/017094, the contents of which are incorporated by reference herein. For example, rapid administration of NAC is the standard of care for preventing hepatic injury in acetaminophen overdose. NAC administered intravenously in dogs has been shown to protect against pulmonary oxygen toxicity and against ischemic and reperfusion damage [Gillissen, A., and Nowak, A., Respir. Med. 92: 609-23, 613 (1998)]. NAC also has anti-inflammatory properties. Id.

Since reactive oxygen species are constantly formed in the lung, and since oxygen metabolites are believed to play a predominant role in the pathogenesis of various pulmonary inflammatory disorders, antioxidant therapy would seem to be a rational approach to take in pulmonary diseases. Patients with acute respiratory distress syndrome (ARDS), idiopathic pulmonary fibrosis (IPF), or chronic obstructive pulmonary disorder (COPD) have been the primary targets for clinical studies evaluating the efficacy of NAC in antioxidant therapy. The results have been, for the most part, inconclusive. For example,

U.S. Pat. No. 5,824,693 discloses a method for treating ARDS and infant respiratory distress syndrome (IRDS), which result in oxidative stress that can damage the cells of the lung. The method increases the intracellular synthesis of glutathione by administering a noncysteine glutathione precursor that will stimulate the intracellular synthesis of glutathione.

Gillissen and Nowak, Respir. Med. 92: 609-23, 614 (1998), who assessed the clinical feasibility of antioxidant therapy with NAC in ARDS, IPF and COPD, reported that improvements in glutathione levels were seen in patients with ARDS and IPF, but not COPD, who received 600-1800 mg NAC given daily by mouth. NAC has been used for over 20 years to treat COPD, a disease not characterized by glutathione deficiency; some studies have demonstrated a beneficial effect, but others have not. Id. at 615.

Cystic fibrosis (CF) is an inherited autosomal recessive disorder. It is one of the most common fatal genetic disorders in the United States, affecting about 30,000 individuals and is most prevalent in the Caucasian population, occurring in one of every 3,300 live births. The gene involved in cystic fibrosis, which was identified in 1989, codes for a protein called the cystic fibrosis transmembrane conductance regulator (CFTR). CFTR is normally expressed by exocrine epithelia throughout the body and regulates the movement of chloride ions, bicarbonate ions and glutathione into and out of cells. In cystic fibrosis patients, mutations in the CFTR gene lead to alterations or total loss of CFTR protein function, resulting in defects in osmolarity, pH and redox properties of exocrine secretions. In the lungs, CF manifests itself by the presence of a thick mucus secretion which clogs the airways. In other exocrine organs, such as the sweat glands, CF may not manifest itself by an obstructive phenotype, but rather by abnormal salt composition of the secretions (hence the clinical sweat osmolarity test used to identify CF in patients).

The predominant cause of illness and death in cystic fibrosis patients is progressive lung disease. The thickness of CF mucus, which blocks the airway passages, is believed to stem from abnormalities in osmolarity of secretions, as well as from the presence of massive amounts of DNA, actin, proteases and prooxidative enzymes originating from a subset of inflammatory cells, called neutrophils. Indeed, CF lung disease is characterized by early, hyperactive neutrophil-mediated inflammatory reactions to both viral and bacterial pathogens.

The hyperinflammatory syndrome of CF lungs has several underpinnings, among which an imbalance between pro-inflammatory chemokines, chiefly interleukin-8 (IL-8), and antiinflammatory cytokines, chiefly IL-10, seems to play a major role. See Chmiel et al. Clin Rev Allergy Immunol. 3(1):5-27 (2002). Besides, chronic oxidative stress in CF patients may severely affect the deformability of blood neutrophils circulating in CF lung capillaries, thereby increasing their recruitment to the lungs. See Hogg, Physiol Rev. 67(4):1249-95 (1987). Chronic oxidative stress in CF is linked to the overwhelming release of oxidants by inflammatory lung neutrophils, and to abnormal antioxidant defenses caused by malabsorption of dietary antioxidant through the gut and a possible defect in GSH efflux. See Wood et al. J. Am. Coll. Nutr. 20 (2 Suppl):157-165 (2001).

The hyperinflammatory syndrome at play in CF lungs may predispose such patients to chronic infections with colonizing bacterial pathogens. The most common bacterium to infect the CF lung is Pseudomonas aeruginosa, a gram-negative microorganism. The lungs of most children with CF become colonized by P. aeruginosa before their third birthday. By their tenth birthday, P. aeruginosa becomes dominant over other opportunistic pathogens. See Gibson et al., Am. J. Respir. Crit Care Med., 168(8): 918-951 (2003). P. aeruginosa infections further exacerbate neutrophilic inflammation, which causes repeated episodes of intense breathing problems in CF patients. Although antibiotics can decrease the frequency and duration of these attacks, the bacterium progressively establishes a permanent residence in CF lungs by switching to a so-called “mucoid”, biofilm form of high resistance and low virulence, which never can be eliminated completely from the lungs. The continuous presence in CF lungs of inflammatory by-products, such as extracellular DNA and elastase, could play a major role in selecting for mucoid P. aeruginosa forms. See Walker et al. Infect Immun. 73(6): 3693-3701 (2005).

Treatments for CF lung disease typically involve antibiotics, anti-inflammatory drugs, bronchodilators, and chest physiotherapy to help fight infection, neutrophilic inflammation and obstruction and clear the airways. Nevertheless, the persistent, viscous and toxic nature of airway secretions in cystic fibrosis lung disease still leads to progressive deterioration of lung function. See Rancourt et al., Am. J. Physiol. Lung Cell Mol. Physiol. 286(5): L931-38 (2004).

N-acetylcysteine (NAC) is a widely used mucolytic drug in patients with a variety of disorders, including cystic fibrosis. See Rochat, et al., J. Cell Physiol. 201(1): 106-16 (2004). It has been hypothesized that NAC works as a mucolytic by rupturing the disulfide bridges of the high molecular weight glycoproteins present in the mucus, resulting in smaller subunits of the glycoproteins and reduced mucous viscosity. Id. To this end, researchers and clinicians have administered NAC to CF patients generally by nebulization, as well as orally. Two placebo-controlled studies have reported beneficial effects of oral NAC treatment on lung function in cystic fibrosis. See G. Stafanger, et al., Eur. Respir. J. 1(2): 161-67 (1988). Active treatment consisted of NAC administered as a 200 mg oral dose three times daily (for patients weighing less than 30 kg) or as a 400 mg oral dose two times daily (for patients weighing more than 30 kg). Ratjen, F., et al., Eur. J. Pediatr. 144(4): 374-78 (1985) reported improvement in some measures of lung function but saw no significant clinical differences between patients treated with oral NAC (200 mg 3 times a day), the secretolytic drug ambroxol (30 mg, three times daily), and placebo. A very short fourth study (2 weeks) failed to find any significant difference between the trial arms. See Gotz et al, Eur. J. Resp. Dis. 61 (Suppl) 111: 122-26 (1980).

Duijvestijin, Y. C. and Brand, P. L. Acta Paediatr. 88(1): 38-41 (1999) observed, however, that despite the fact that NAC commonly is used in CF, there is remarkably little published data on its effects. They tested their hypothesis that NAC's antioxidant properties could be useful in preventing decline of lung function (defined as forced expiratory volume in one second, or FEV₁, meaning the volume of air that can be exhaled during the first second of a forced exhalation, which is a reflection of the flow of air in the large airways of the lung) in cystic fibrosis by performing a systematic review of the literature to evaluate whether published evidence supports the use of NAC administered orally or by nebulization to improve lung function in patients with cystic fibrosis. They identified 23 papers, the majority of which were uncontrolled clinical observations, of which only three randomized controlled trials on nebulized NAC were found. None of these studies showed a statistically significant or clinically relevant beneficial effect of NAC aerosol. They found a small beneficial effect of doubtful clinical relevance of oral NAC on FEV₁ in CF. Although they suggested that the effects of long-term treatment with oral NAC on lung function in CF should be investigated, they concluded that there is no evidence supporting the use of N-acetylcysteine in cystic fibrosis.

Despite these findings, redox-based therapy is an attractive idea for CF, since redox imbalance is a well-recognized aspect of the disease, yet seldom considered as a therapeutic target. See Cantin, Curr Opin Pulm Med. 10(6):531-6 (2004). Systemic oxidative stress can affect blood neutrophils by lowering their intracellular GSH levels. This in turn renders lungs more prone to air trapping and dysfunction. See Hogg, Physiol Rev. 67(4):1249-95 (1987). Besides, systemic oxidative stress can alter the chemokine/cytokine balance, favoring inflammation, which systemic NAC treatment can help alleviate. See Zafarullah et al. Cell Mol Life Sci. 60(1):6-20 (2003).

We therefore have investigated whether NAC in high doses could counter systemic oxidative stress/redox imbalance and inhibit inflammation when administered orally to CF patients. Our strategy is to target blood neutrophils before they reach the lung, a strategy that has not been tested in CF. Thus, our focus is on the inflammatory and redox aspects of CF lung disease, which are major contributors to the progression of the disease. This work was supported by the Cystic Fibrosis Foundation.

### SUMMARY OF THE INVENTION

The present invention relates to N-acetylcysteine compositions and methods to treat lung inflammation and redox imbalance conditions in human cystic fibrosis patients. The present invention provides a method of treating a lung inflammation condition in cystic fibrosis patients, the method comprising the step of administering to a patient in need thereof a pharmaceutical composition comprising an inflammation-reducing amount of N-acetylcysteine, a pharmaceutically acceptable salt of N-acetylcysteine, or a pharmaceutically acceptable derivative of N-acetylcysteine, and a pharmaceutically acceptable carrier, thereby modulating the lung inflammation. According to one embodiment, the lung inflammation condition is acute or chronic. In another embodiment, in step (a) of the method, the pharmaceutical composition is administered systemically by a route selected from the group consisting of orally, buccally, topically, by inhalation, by insufflation, parenterally and rectally. In another embodiment, the pharmaceutical composition is administered orally. In another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition administered orally is about 1.8 grams per day to about 6 grams per day, and less than or equal to 70 mg/kg/d. In another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition administered orally is at least about 1800 mg per day and less than or equal to 70 mg/kg/d. In another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 70 mg/kg/d. In another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition administered orally is at least about 3000 mg per day and less than or equal to 70 mg/kg/d. In another embodiment, the pharmaceutical composition is administered parenterally. In another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition administered parenterally is about 200 mg NAC to about 20000 mg NAC per dosage unit. In another embodiment, the method further comprises the step of administering a pharmaceutically effective amount of a cystic fibrosis therapeutic agent. In another embodiment, the cystic fibrosis therapeutic agent is at least one agent selected from the group consisting of an anti-infective agent, a bronchodilating agent, and an anti-inflammatory agent. In another embodiment, the method further comprises the step of administering a respiratory therapy to the patient. In another embodiment, the method further comprises the step of administering a rehabilitation therapy to the patient. In another embodiment, the method further comprises the step of monitoring lung function of the patient. In another embodiment, the method further comprises the step of monitoring the lung inflammation by a method comprising the steps of: collecting a sample of blood or sputum from the patient; and determining a measure of inflammatory activity in the blood or sputum collected from the patient. In another embodiment, the measure of inflammatory activity in the sample of blood is at least one measure selected from the group consisting of a plasma level of neutrophil elastase activity and a plasma level of interleukin-8 activity. In another embodiment, the measure of inflammatory activity in the sample of sputum is at least one measure selected from the group consisting of a count of live leukocytes, a count of live neutrophils, a ratio of neutrophils to total leukocytes; a sputum level of neutrophil elastase activity and a sputum level of interleukin-8 activity.

The present invention further provides a method of treating a redox imbalance condition in cystic fibrosis patients, the method comprising the step of administering to a patient in need thereof a pharmaceutical composition comprising a redox-balancing amount of N-acetylcysteine, a pharmaceutically acceptable salt of N-acetylcysteine, or a pharmaceutically acceptable derivative of N-acetylcysteine, and a pharmaceutically acceptable carrier, thereby modulating the redox imbalance condition. According to one embodiment, the pharmaceutical composition is administered systemically by a route selected from the group consisting of orally, buccally, parenterally, topically, by inhalation, by insufflation, and rectally. According to another embodiment, the pharmaceutical composition is administered orally. According to another embodiment, the redox-balancing amount of N-acetylcysteine, a pharmaceutically acceptable salt of N-acetylcysteine, or a pharmaceutically acceptable derivative of N-acetylcysteine in the pharmaceutical composition administered orally is about 1.8 grams per day to about 6 grams per day and less than or equal to 70 mg/kg/d. According to another embodiment, the redox-balancing amount of N-acetylcysteine, a pharmaceutically acceptable salt of N-acetylcysteine, or a pharmaceutically acceptable derivative of N-acetylcysteine in the pharmaceutical composition administered orally is at least about 1800 mg per day and less than or equal to 70 mg/kg/d. According to another embodiment, the redox-balancing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 70 mg/kg/d. According to another embodiment, the redox-balancing amount of N-acetylcysteine, a pharmaceutically acceptable salt of N-acetylcysteine, or a pharmaceutically acceptable derivative of N-acetylcysteine in the pharmaceutical composition administered orally is at least about 3000 mg per day and less than or equal to 70 mg/kg/d. In another embodiment, the pharmaceutical composition is administered parenterally. In another embodiment, the redox-balancing amount of N-acetylcysteine, a pharmaceutically acceptable salt of N-acetylcysteine, or a pharmaceutically acceptable derivative of N-acetylcysteine in the pharmaceutical composition administered parenterally is about 200 mg NAC to about 20000 mg NAC per dosage unit. According to another embodiment, the method further comprises the step of administering a pharmaceutically effective amount of a cystic fibrosis therapeutic agent. According to another embodiment, the cystic fibrosis therapeutic agent is at least one agent selected from the group consisting of an anti-infective agent, a bronchodilating agent, and an anti-inflammatory agent. According to another embodiment, the method further comprises the step of administering a respiration therapy to the patient. According to another embodiment, the method further comprises the step of administering a rehabilitative therapy to the patient. According to another embodiment, the method further comprises the step of monitoring lung function of the patient. According to another embodiment, the method further comprises the step of monitoring the redox imbalance in cystic fibrosis patients by a method comprising the steps of collecting a sample of blood or sputum from the patient; and determining a measure of redox balance in the sample of blood or sputum. According to another embodiment, the measure of redox balance in the sample of blood is at least one measure selected from the group consisting of a level of reduced glutathione in whole blood and a level of reduced glutathione in live blood neutrophils.

Moreover, the present invention provides a pharmaceutical kit for treating a lung inflammation condition in cystic fibrosis patients, the kit comprising a first container containing a pharmaceutically effective amount of a cystic fibrosis therapeutic agent, and a second container containing a pharmaceutical composition comprising an inflammation-reducing amount of N-acetylcysteine, a pharmaceutically acceptable salt of N-acetylcysteine, or a pharmaceutically acceptable derivative of N-acetylcysteine, and a pharmaceutically acceptable carrier. According to one embodiment, the pharmaceutical composition in the second container is to be administered systemically by a route selected from the group consisting of orally, buccally, parenterally, topically, by inhalation, by insufflation, or rectally. According to another embodiment, the pharmaceutical composition in the second container is administered orally. According to another embodiment, the pharmaceutical composition to be administered orally that is in the second container is in an oral form selected from the forms consisting of a tablet, a troche, a lozenge, an aqueous suspension, an oily suspension, a dispersible powder, a dispersible granule, an emulsion, a hard capsule, a soft capsule, a syrup, and an elixir. According to another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is about 1.8 grams per day to about 6 grams per day, and less than or equal to 70 mg/kg/d. According to another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 1800 mg per day and less than or equal to 70 mg/kg/d. According to another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 2400 mg per day and less than or equal to 70 mg/kg/d. According to another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 3000 mg per day and less than or equal to 70 mg/kg/d. In another embodiment, the pharmaceutical composition in the second container is to be administered parenterally. In another embodiment, the inflammation-reducing amount of N-acetylcysteine, a pharmaceutically acceptable salt of N-acetylcysteine, or a pharmaceutically acceptable derivative of N-acetylcysteine in the pharmaceutical composition to be administered parenterally that is in the second container is about 200 mg NAC to about 20000 mg NAC per dosage unit. According to another embodiment, the cystic fibrosis therapeutic agent in the first container is at least one agent selected from the group consisting of an anti-infective agent, a bronchodilating agent, and an anti-inflammatory agent.

In addition, the present invention provides a pharmaceutical kit for treating a redox imbalance condition in cystic fibrosis patients, the kit comprising a first container containing a pharmaceutically effective amount of a cystic fibrosis therapeutic agent, and a second container containing a pharmaceutical composition comprising a redox-balancing amount of N-acetylcysteine, a pharmaceutically acceptable salt of N-acetylcysteine, or a pharmaceutically acceptable derivative of N-acetylcysteine, and a pharmaceutically acceptable carrier. According to one embodiment, the pharmaceutical composition in the second container is to be administered systemically by a route selected from the group consisting of orally, buccally, parenterally, topically, by inhalation, by insufflation, or rectally. In another embodiment, the pharmaceutical composition in the second container is to be administered orally. In another embodiment, the pharmaceutical composition to be administered orally that is in the second container is in a form selected from the forms consisting of a tablet, a troche, a lozenge, an aqueous suspension, an oily suspension, a dispersible powder, a dispersible granule, an emulsion, a hard capsule, a soft capsule, a syrup, and an elixir. In another embodiment, the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is about 1.8 grams per day to about 6 grams per day and less than or equal to 70 mg/kg/d. In another embodiment, the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 1800 mg per day and less than or equal to 70 mg/kg/d. In another embodiment, the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 2400 mg per day and less than or equal to 70 mg/kg/d. In another embodiment, the redox-balancing amount of N-acetylcysteine, a pharmaceutically acceptable salt of N-acetylcysteine, or a pharmaceutically acceptable derivative of N-acetylcysteine in the pharmaceutical composition to be delivered orally that is in the second container is at least about 3000 mg per day and less than or equal to 70 mg/kg/d. In another embodiment, the pharmaceutical composition is administered parenterally. In another embodiment, the redox-balancing amount of N-acetylcysteine, a pharmaceutically acceptable salt of N-acetylcysteine, or a pharmaceutically acceptable derivative of N-acetylcysteine in the pharmaceutical composition to be administered parenterally that is in the second container is about 200 mg NAC to about 20000 mg NAC per dosage unit. In another embodiment, the cystic fibrosis therapeutic agent in the first container is at least one agent selected from the group consisting of an anti-infective agent, a bronchodilating agent, and an anti-inflammatory agent.

### DETAILED DESCRIPTION OF THE INVENTION

The term “condition”, as used herein, refers to a variety of health states and is meant to include disorders or diseases caused by any underlying mechanism or disorder, injury, and the promotion of healthy tissues and organs. The term “disease” or “disorder” as used herein refers to an impairment of health or a condition of abnormal functioning. The term “syndrome,” as used herein, refers to a pattern of symptoms indicative of some disease or condition.

As used herein, the term “modulate” or “modulating” refers to adjusting, changing, or manipulating the function or status of at least one of redox balance or inflammation in cystic fibrosis. Such modulation may be any change, including an undetectable change. In one embodiment of the present invention, a method of treating an inflammation in cystic fibrosis patients comprises the steps of administering to a patient in need thereof a composition comprising an inflammation-reducing amount of NAC, a pharmaceutically acceptable salt of NAC, or a pharmaceutically acceptable derivative of NAC, and a pharmaceutically acceptable carrier and a pharmaceutically acceptable carrier, thereby modulating the inflammation.

As used herein the term “treating” includes abrogating, substantially slowing or reversing the progression of a condition, substantially ameliorating clinical or symptoms of a condition, and substantially preventing the appearance of clinical or symptoms of a condition.

The term “inflammation” as used herein refers to the physiologic process by which vascularized tissues respond to injury. See, e.g., FUNDAMENTAL IMMUNOLOGY, 4^{th} Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses. The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited, to pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents. Chronic inflammation takes over when acute inflammation persists, either through incomplete clearance of the initial inflammatory agent or as a result of multiple acute events occurring in the same location. The term “chronic inflammation” as used herein refers to inflammation that is of longer duration and which has a vague and indefinite termination. Chronic inflammation, which includes the influx of lymphocytes and macrophages and fibroblast growth, may result in tissue scarring at sites of prolonged or repeated inflammatory activity.

Intracellular redox status plays a critical role in cell function. The term “oxidative stress” as used herein refers to a condition caused by an imbalance between reactive oxygen species and the antioxidant defense mechanisms of a cell, leading to an excess production of oxygen metabolites. Skaper, et al., Free Radical Biol. & Med. 22(4): 669-678 (1997). The term “redox imbalance” as used herein refers to the imbalance between reactive oxygen species and the antioxidant defense mechanisms of a cell. In another embodiment of the present invention, a method of treating a redox imbalance condition in cystic fibrosis patients comprises the steps of administering to a patient in need thereof a composition comprising a redox-balancing amount of NAC, a pharmaceutically acceptable salt of NAC, or a pharmaceutically acceptable derivative of NAC, and a pharmaceutically acceptable carrier and a pharmaceutically acceptable carrier, thereby modulating the redox imbalance condition.

As used herein the terms “inflammation-reducing amount,” “redox imbalance adjusting amount”, or “pharmaceutically effective amount” refer to the amount of the compositions of the invention that result in a therapeutic or beneficial effect following its administration to a subject. The inflammation-reducing, redox imbalance adjusting or pharmaceutical effect can be curing, minimizing, preventing or ameliorating a disease or disorder, or may have any other anti-inflammatory, redox balancing or pharmaceutical beneficial effect. The concentration of the substance is selected so as to exert its inflammation-reducing, redox balancing, or pharmaceutical effect, but low enough to avoid significant side effects within the scope and sound judgment of the physician. The effective amount of the composition may vary with the age and physical condition of the biological subject being treated, the severity of the condition, the duration of the treatment, the nature of concurrent therapy, the specific compound, composition or other active ingredient employed, the particular carrier utilized, and like factors.

A skilled artisan can determine a pharmaceutically effective amount of the inventive compositions by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED50. The use of the term “individual” distinguishes the ED50 based on the intensity of effect as used herein from the median effective dose, also abbreviated ED50, determined from frequency of response data in a population. “Efficacy” as used herein refers to the property of the compositions of the present invention to achieve the desired response, and “maximum efficacy” refers to the maximum achievable effect. The amount of the NAC compounds in the compositions of the present invention which will be effective in the treatment of a particular disorder or condition will depend on the nature of the disorder or condition, and can be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN'S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed in the formulations of the present invention also will depend on the route of administration and the seriousness of the disease or disorder, and should be decided according to the judgment of the practitioner and each patient's circumstances.

It is preferred that the pharmaceutical compositions according to the present invention contain from about at least about 200 mg NAC to about 2000 mg NAC per dosage unit for oral administration. Thus, the minimum pharmaceutically effective amount of NAC, pharmaceutically effective salts of NAC, or pharmaceutically acceptable NAC derivatives per dosage unit for oral administration according to the present invention is at least about: 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg, and the maximum pharmaceutically effective amount of NAC, pharmaceutically effective salts of NAC, or pharmaceutically acceptable NAC derivatives per dosage unit for oral administration according to the present invention is no more than about: 2000 mg, 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, 500 mg, 400 mg, 300 mg, or 200 mg. It is preferred that the pharmaceutical compositions according to the present invention contain from about at least about 200 mg NAC to about 20000 mg NAC per dosage unit for parenteral administration at the physician's discretion. The minimum pharmaceutically effective amount of NAC, pharmaceutically effective salts of NAC, or pharmaceutically acceptable NAC derivatives per dosage unit for parenteral administration according to the present invention is at least about: 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 11000 mg, 12000 mg, 13000 mg, 14000 mg, 15000 mg, 16000 mg, 17000 mg, 18000 mg, 19000 mg, or 20,000 mg, and the maximum pharmaceutically effective amount of NAC, pharmaceutically effective salts of NAC, or pharmaceutically acceptable NAC derivatives per dosage unit for parenteral administration according to the present invention is no more than about: 20000 mg, 19000 mg, 18000 mg, 17000 mg, 16000 mg, 15000 mg, 14000 mg, 13000 mg, 12000 mg, 11000 mg, 10000 mg, 9500 mg, 9000 mg, 8500 mg, 8000 mg, 7500 mg, 7000 mg, 6500 mg, 6000 mg, 5500 mg, 5000 mg, 4500 mg, 4000 mg, 3500 mg, 3000 mg, 2500 mg, 2000 mg, 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, 500 mg, 400 mg, 300 mg, or 200 mg. Usual dosage should be about 1.8 grams per day (“g/d”) to about 6.0 g/d (i.e., a minimum of about: 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 g/d and a maximum of about: 6.0, 5.8, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 2.9, 2.8, 2.7, 2.6, 2.5, 2.4, 2.3, 2.2, 2.1, 2.0, 1.9, or 1.8 g/d), not to exceed about 70 mg per kg per day (“mg/kg/d”).

The unit oral dose of NAC usually will comprise at least about 200 mg (for pediatric doses), usually at least about 600 mg (for adult doses); and usually not more than about 2000 mg at the physician's discretion, from a minimum of one to a maximum of six daily intakes. Patients on therapy known to deplete cysteine/glutathione or produce oxidative stress may benefit from higher amounts of NAC.

The terms “drug carrier”, “carrier”, or “vehicle” as used herein refers to carrier materials suitable for NAC administration. As used herein, the terms “carrier” and “pharmaceutical carrier” refer to a pharmaceutically acceptable inert agent or vehicle for delivering one or more active agents to a mammal, and often is referred to as “excipient.” As used herein the term. “a pharmaceutically acceptable carrier” refers to any substantially non-toxic carrier conventionally useable for NAC administration in which NAC will remain stable and bioavailable. The (pharmaceutical) carrier must be of sufficiently high purity and of sufficiently low toxicity to render it suitable for administration to the mammal being treated. The (pharmaceutical) carrier further should maintain the stability and bioavailability of an active agent, e.g., a signal transduction modulator compound of the present invention. The (pharmaceutical) carrier can be liquid or solid and is selected, with the planned manner of administration in mind, to provide for the desired bulk, consistency, etc., when combined with an active agent and other components of a given composition. The (pharmaceutical) carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable (pharmaceutical) carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like. Compositions of the present invention that are for cutaneous administration, such as topical (i.e., local), can include (pharmaceutical) carriers such as sterile and non-sterile aqueous solutions, non-aqueous solutions in common solvents such as alcohols, or solutions of NAC in liquid or solid oil bases. Such (pharmaceutical) carrier solutions also can contain buffers, diluents and other suitable additives. Compositions of the present invention that are for parenteral administration of the signal transduction modulator compound, such as intramuscular or subcutaneously, can include (pharmaceutical) carriers such as sterile aqueous solutions, non-aqueous solutions in common solvents such as alcohols, or solutions of NAC in a liquid oil base.

In some embodiments, the carrier of the composition of the present invention includes a release agent such as sustained release or delayed release carrier. In such embodiments, the carrier can be any material capable of sustained or delayed release of NAC to provide a more efficient administration, e.g., resulting in less frequent and/or decreased dosage of NAC, improve ease of handling, and extend or delay effects on diseases, disorders, conditions, syndromes, and the like, being treated, prevented or promoted. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

Regular, routine treatment to keep secretions cleared and prevent infection is very important in CF because respiratory complications are the leading cause of morbidity and mortality in CF patients. Thick mucus blocks the bronchial tubes in the lungs leading to inflammation and recurrent infections. With each infection, more damage or scarring occurs, causing lung function to progressively worsen. Known techniques used in the art to monitor lung function include, but are not limited to, spirometry, which provides information about airflow limitation and lung volumes; plethysmography, which provides information about airway resistance, total lung size, and trapped gas; transfer factor, which provides information about alveolar function; gas washout tests, which provide information about gas mixing, small airway function, and heterogeneous changes in compliance; computational tomography, which provides information about large and small airway deterioration; and oscillometry, which may provide information about small airways.

In another embodiment of the present invention, compositions and methods of the present invention can be used in combination with known cystic fibrosis therapeutic agents, provided that they are compatible with each other. “Compatible” as used herein means that the compositions and methods of the present invention are capable of being combined with existing therapies in a manner such that there is no interaction that would substantially reduce the efficacy of either the compositions or methods of the present invention or the therapies under ordinary use conditions.

For example, existing cystic fibrosis therapeutic agents that may be combined with the compositions and methods of the present invention include, but are not limited to, anti-infective agents, bronchodilating agents, and anti-inflammatory agents.

Lung and airway infections in cystic fibrosis can be treated with potent anti-infective agents, including antibiotics, to improve lung function, reduce days spent in the hospital and to reduce use of intravenous antibiotics to reduce bacterial levels in the lungs. Inhaled antibiotics also are used to prevent lung infections that may lead to hospitalization.

To minimize certain side effects, bronchodilating agents often are used along with inhaled antibiotics. Bronchodilating agents are used widely for treating a variety of obstructive lung diseases, including cystic fibrosis. They relax smooth muscle in the small airways of the lungs, which dilates the airways and makes breathing easier, particularly when airways are narrowed by inflammation. Inhaled bronchodilator medications used in asthma, such as albuterol, have improved breathing in some people with cystic fibrosis. When used to treat cystic fibrosis, bronchodilating agents are usually given through a nebulizer or with a handheld inhaler. Airway dilatation before physiotherapy helps the cystic fibrosis patient to clear chest secretions.

Nonsteroidal anti-inflammatory agents reduce inflammation and pain. Cystic fibrosis patients often have persistent lung inflammation which becomes part of the cycle of continued lung damage in these patients. Anti-inflammatory medications, such as ibuprofen, in some patients with CF help to reduce this inflammation. In some children, anti-inflammatory medications can significantly slow the progression of lung disease and improve breathing.

In another embodiment of the present invention, compositions and methods of the present invention can be used in combination with known cystic fibrosis therapies, provided that they are compatible with each other. The terin “respiratory therapy” as used herein refers to chest physiotherapy, which is used to help clear excess mucus out of the lungs. To perform chest physiotherapy, a patient is placed in various positions allowing major segments of the lungs to point downward and then clapped firmly over chest and back on part of the lung segment to shake the mucus loose. Once loosened, the mucus will fall to the large airways, where it can be coughed out. Chest physiotherapy can be time-consuming since 3-5 minutes is spent clapping over 10-12 lung segments. It also is difficult for patients to perform on themselves and usually requires a skilled caregiver.

The term “rehabilitative therapy” refers to a therapy designed to help cystic fibrosis patients use their energy more efficiently, i.e., in a way that requires less oxygen. Rehabilitative therapy improves shortness of breath and overall survival, especially in those with advanced disease.

It is preferred that the NAC be substantially free of sulfones or other chemicals that interfere with the metabolism of any co-administered drug in its bioactive form. It is also preferred that the NAC be substantially free of its oxidized form, di-N-acetylcysteine, and that the composition should be prepared in a manner that substantially prevents oxidation of the NAC during preparation or storage.

The effectiveness of NAC depends on the presence of the reduced form, which may, for example, liberate the reduced form of glutathione from homo- and hetero-disulfide derivatives in thiol-disulfide exchange reactions. A typical unit dosage may be a solution suitable for oral or intravenous administration; an effervescent tablet suitable for dissolving in water, fruit juice, or carbonated beverage and administered orally; a tablet taken from two to six times daily, or one time-release capsule or tablet taken several times a day and containing a proportionally higher content of active ingredient, etc. The time-release effect may be obtained by capsule materials that dissolve at different pH values, by capsules that release slowly by osmotic pressure, or by any other known means of controlled release. Unit dosage forms may be provided wherein each dosage unit, for example, teaspoonful, tablespoonful, gel capsule, tablet or suppository, contains a predetermined amount of the compositions of the present invention. Similarly, unit dosage forms for injection or intravenous administration may comprise the compound of the present invention in a composition as a solution in sterile water, normal saline or another pharmaceutically acceptable carrier. The specifications for the unit dosage forms of the present invention depend on the effect to be achieved and the intended recipient.

Over-the-counter NAC can be variably produced and packaged. Because the production and packaging methods generally do not guard against oxidation, the NAC can be significantly contaminated with bioactive oxidation products. These may be particularly important in view of data indicating that the oxidized form of NAC has effects counter to those reported for NAC and is bioactive at doses roughly 10-100 fold less than NAC. See Sarnstrand et al J. Pharmacol. Exp. Ther. 288:1174-84 (1999).

The distribution of the oxidation states of NAC as a thiol and disulfide depends on the oxidation/reduction (redox) potential. The half-cell potential obtained for the NAC thiol/disulfide pair is about +63 mV, indicative of its strong reducing activity among natural compounds [see Noszal et al. J. Med. Chem. 43:2176-2182 (2000)]. In a preferred embodiment of the invention, the preparation and storage of the formulation is performed in such a way that the reduced form of NAC is the primary form administered to the patient. Maintaining NAC containing formulations in solid form is preferable for this purpose. When in solution, NAC containing formulations are preferably stored in a brown bottle that is vacuum sealed. Storage in cool dark environments is also preferred.

The determination of reduced and oxidized species present in a sample may be determined by various methods known in the art, including, but not limited to, for example, capillary electrophoresis, and high performance liquid chromatography as described by Chassaing et al. J. Chromatogr. B. Biomed. Sci. Appl. 735(2):219-27 (1999).

The compositions of the present invention may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired.

The compositions of the present invention may be in a form suitable for oral use, for example, as tablets, troches, lozenges, aqueous or oily suspensions, dispersible powders or granules, emulsions, hard or soft capsules or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient(s) in admixture with non-toxic pharmaceutically-acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They also may be coated for controlled release.

Compositions of the present invention also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the present invention may be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the present invention may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the present invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, for example, sweetening, flavoring and coloring agents also may be present.

The compositions of the invention also may be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example, soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

The compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

For buccal administration, the compositions of the present invention may take the form of tablets or lozenges formulated in a conventional manner.

The compositions of the present invention may be in the form of a sterile injectable aqueous or oleaginous suspension. The term “parenteral” as used herein includes subcutaneous injections, intravenous, intramuscular, intrasternal injection, or infusion techniques. Injectable preparations, such as sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are employed conventionally as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The term “topical” refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably. For the purpose of this application, topical applications shall include mouthwashes and gargles.

Topical administration also may involve the use of transdermal administration such as transdermal patches or iontophoresis devices, which are prepared according to techniques and procedures well known in pharmacology. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches suitable for use in the present invention include, but are not limited to (1) the matrix patch; (2) the reservoir patch; (3) the multi-laminate drug-in-adhesive patch; and (4) the monolithic drug-in-adhesive patch TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

The compositions of the present invention may be in the form of a dispersible dry powder for pulmonary delivery. Dry powder compositions may be prepared by processes known in the art as disclosed in U.S. Pat. No. 6,921,527, the disclosures of which are incorporated by reference. Spray drying, for example, is a process in which a homogeneous aqueous mixture of drug and the carrier is introduced via a nozzle (e.g., a two fluid nozzle), spinning disc or an equivalent device into a hot gas stream to atomize the solution to form fine droplets. The aqueous mixture may be a solution, suspension, slurry, or the like, but needs to be homogeneous to ensure uniform distribution of the components in the mixture and ultimately the powdered composition. The solvent, generally water, rapidly evaporates from the droplets producing a fine dry powder having particles from about 1 μm to 5 μm in diameter. The spray drying is done under conditions that result in a substantially amorphous powder of homogeneous constitution having a particle size that is respirable, a low moisture content and flow characteristics that allow for ready aerosolization. Preferably the particle size of the resulting powder is such that more than about 98% of the mass is in particles having a diameter of about 10 μm or less with about 90% of the mass being in particles having a diameter less than 5 μm. Alternatively, about 95% of the mass will have particles with a diameter of less than 10 μm with about 80% of the mass of the particles having a diameter of less than 5 μm. Dry powder compositions also may be prepared by lyophilization and jet milling, as disclosed in International Patent Publication No. WO 91/16038, the disclosure of which are incorporated by reference.

The term “dispersibility” or “dispersible” means a dry powder having a moisture content of less than about 10% by weight (% w) water, usually below about 5% w and preferably less than about 3% w; a particle size of about 1.0-5.0 μm mass median diameter (MMD), usually 1.0-4.0 μm MMD, and preferably 1.0-3.0 μm MMD; a delivered dose of about >30%, usually >40%, preferably >50%, and most preferred >60%; and an aerosol particle size distribution of about 1.0-5.0 μm mass median aerodynamic diameter (MMAD), usually 1.5-4.5 μm MMAD, and preferably 1.5-4.0 μm MMAD. Methods and compositions for improving dispersibility are disclosed in U.S. application Ser. No. 08/423,568, filed Apr. 14, 1995, the disclosure of which is hereby incorporated by reference.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 microns (μm) in diameter with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content such that the particles are readily dispersible in an inhalation device to form an aerosol. This moisture content is generally below about 10% by weight (% w) water, usually below about 5% w and preferably less than about 3% w.

The amount of the pharmaceutically acceptable carrier is that amount needed to provide the necessary stability, dispersibility, consistency and bulking characteristics to ensure a uniform pulmonary delivery of the composition to a subject in need thereof. Numerically the amount may be from about 0.05% w to about 99.95% w, depending on the activity of the drug being employed. Preferably about 5% w to about 95% will be used. The carrier may be one or a combination of two or more pharmaceutical excipients, but generally will be substantially free of any “penetration enhancers.” Penetration enhancers are surface active compounds which promote penetration of a drug through a mucosal membrane or lining and are proposed for use in intranasal, intrarectal, and intravaginal drug formulations. Exemplary penetration enhancers include bile salts, e.g., taurocholate, glycocholate, and deoxycholate; fusidates, e.g., taurodehydrofusidate; and biocompatible detergents, e.g., Tweens, Laureth-9, and the like. The use of penetration enhancers in formulations for the lungs, however, is generally undesirable because the epithelial blood barrier in the lung can be adversely affected by such surface active compounds. The dry powder compositions of the present invention are readily absorbed in the lungs without the need to employ penetration enhancers.

The types of pharmaceutical excipients that are useful as carriers for pulmonary delivery include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable for pulmonary delivery include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. A preferred group of carbohydrates includes lactose, trehalose, raffinose, maltodextrins, and mannitol. Suitable polypeptides include aspartame. Amino acids include alanine and glycine, with glycine being preferred.

Additives, which are minor components of the composition for pulmonary delivery, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate is preferred.

The composition of the present invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. No. 4,227,522; U.S. Pat. No. 4,192,309; and U.S. Pat. No. 4,105,027. Suitable containers also include those used in conjunction with Glaxo's Ventolin® Rotohaler brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

The compositions of the present invention may be in the form of suppositories for rectal administration of the composition. These compositions can be prepared by mixing the drug with a suitable nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

The therapeutically active agent of the present invention can be formulated per se or in salt form. The term “pharmaceutically acceptable salts” refers to nontoxic salts of NAC. Pharmaceutically acceptable salts include, but are not limited to, those formed with free amino groups such as those derived from hydrochloric, phosphoric, sulfuric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The term “pharmaceutically acceptable N-acetylcysteine derivative” as used herein refers to a pharmaceutically acceptable compound formed from N-acetylcysteine or a pharmaceutically acceptable compound that can be imagined to arise from N-acetylcysteine if one atom is replaced with another atom or group of atoms.

Additional compositions of the present invention can be readily prepared using technology which is known in the art such as described in Remington's Pharmaceutical Sciences, 18^{th} or 19^{th} editions, published by the Mack Publishing Company of Easton, Pa., which is incorporated herein by reference.

The present invention further provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

For example, in one embodiment, a pharmaceutical kit for treating lung inflammation in cystic fibrosis patients according to the present invention includes a first container containing a pharmaceutically effective amount of a cystic fibrosis therapeutic agent and a second container containing a pharmaceutical composition comprising an inflammation-reducing amount of N-acetylcysteine, a pharmaceutically acceptable salt of N-acetylcysteine, or a pharmaceutically acceptable derivative of N-acetylcysteine, and a pharmaceutically acceptable carrier. In another embodiment, a pharmaceutical kit for treating redox imbalance in cystic fibrosis patients according to the present invention includes a first container containing a pharmaceutically effective amount of a cystic fibrosis therapeutic agent and a second container containing a pharmaceutical composition comprising a redox-balancing amount of N-acetylcysteine, a pharmaceutically acceptable salt of N-acetylcysteine, or a pharmaceutically acceptable derivative of N-acetylcysteine, and a pharmaceutically acceptable carrier. In yet another embodiment, a pharmaceutical kit for treating inflammation and redox imbalance in cystic fibrosis patients according to the present invention includes a first container filled with a pharmaceutically effective amount of a cystic fibrosis therapeutic agent and a second container filled with a pharmaceutical composition comprising an inflammation-reducing and redox-balancing amount of N-acetylcysteine, a pharmaceutically acceptable salt of N-acetylcysteine, or a pharmaceutically acceptable derivative of N-acetylcysteine, and a pharmaceutically acceptable carrier.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Treatment of Cystic Fibrosis Patients with Oral N-acetylcysteine

A phase I trial of high-dose oral N-acetylcysteine (NAC) in CF has been completed. This CF Foundation-sponsored dose-escalation safety pilot study was designed to assess the dose of oral NAC that can be used safely in order to replenish glutathione (GSH) stores in subjects with CF, with the objectives of restoring a proper redox balance and limiting lung inflammation in patients.

Safety was excellent with all doses tested (about 1.8 g/d (cohort 1), about 2.4 g/d (cohort 2) and about 3.0 g/d (cohort 3), divided in three equal doses usually taken at breakfast, lunch, and dinner, for 4 weeks (N=6 in each cohort). No clinical adverse effect was identified based on physical examination, Complete Blood Count (“CBC”, meaning a series of tests to examine components of the blood that are useful in diagnosing certain health problems and in following the effects of treatment), laboratory tests, and the CF patient's quality of life (“QOL”). Very mild and infrequent drug-related adverse effects were reported in 6 out of 18 patients (Table 1): heartburn (N=4), nausea (N=1), bad taste (N=1). Doses of about 2.4 g/d and about 3.0 g/d had less reported adverse effects than a dose of about 1.8 g/d. Treatment compliance was high (93±1%) and not impacted by drug-related adverse effects (P>0.7) or dose (P>0.3).

With regards to efficacy, very significant positive effects of the treatment were observed. These positive effects (Table 2) included amelioration of: 1—Whole blood GSH (+11%, P=0.03), as measured by high performance liquid chromatography (HPLC) and blood neutrophil GSH (+17%, P=0.03), as measured by flow cytometry; 2—Live sputum leukocyte (−21%, P=0.03) and neutrophil (−25%, P=0.02) counts, as measured by microscopy and sputum elastase activity (−44%, P=0.02), as measured by kinetic spectrophotometry; and 3—Perceived weight gain (P=0.01), as measured by the CF QOL

After excluding three patients without basal lung inflammation (total live leukocytes in sputum in normal range [<0.9, Log₁₀ scale], treatment effects even were more pronounced: 1—Whole blood GSH (+14%, P=0.02) and blood neutrophil GSH (+25%, P=0.003); 2—Live sputum leukocyte (−28, P=0.005) and neutrophil (−32%, P=0.003) counts and sputum elastase activity (−46%, P=0.02), as well as % neutrophils in sputum (−9%, P=0.04) and sputum IL-8 (−25%, P=0.02); 3—Perceived weight gain, on the other hand, was less significantly altered (P=0.05) when excluding the three CF patients without basal lung inflammation

The three dose cohorts were not significantly different with regards to most outcome measurements, but the second and third dose cohorts (about 2.4 g/d and about 3.0 g/d) performed slightly better overall than the first (about 1.8 g/d). As expected with short-term treatment (meaning 4 weeks), Pulmonary Function Testing results (“PFT”) were not changed.

1. Data Acquisition

Data acquisition was completed very satisfactorily for clinical assessment, clinical laboratory tests and research tests. Only one patient in cohort 1 failed to give enough blood to perform both clinical laboratory and research tests so that only the latter were performed.

2. Safety, Adverse Effects and Compliance

Safety assessment did not raise any particular concern. Sputum induction was well tolerated. No clinical adverse effect of treatment was identified based on physical examination, CBC, common laboratory tests and CF QOL (no diarrhea or vomiting recorded). High-dose oral NAC thus was very well tolerated, with only very mild drug-related adverse effects (Table 1, below). Adverse effects were not correlated with dose, patient age, gender, P. aeruginosa status or other parameters. Compliance was excellent, averaging 93±1% (mean±SE) overall, was not influenced by the advent of reported adverse effects, and did not differ between the three dose cohorts. Therefore, dose escalation proceeded from cohort 1 to 3 with no safety concerns.

TABLE 1
Safety and compliance

| | Subject information | | | | | | Adverse effects | | |
|---|---|---|---|---|---|---|---|---|---|
| Trial | | Age | | | Compliance | Clinical | Patient | Duration | Probable |
| ID | Cohort | (yrs) | Gender | P. aeruginosa | (%) | monitoring | reporting | (days) | cause(s) |
| 001 | 1 | 11 | F | N | 88 | None | Headache | 1 | Dehydration |
| 002 | 1 | 11 | F | Y | 93 | None | Increased cough, | 9 | Infection |
| | | | | | | | sputum; | | |
| | | | | | | | decreased peak | | |
| | | | | | | | flow and | | |
| | | | | | | | exercise | | |
| | | | | | | | tolerance | | |
| 003 | 1 | 40 | F | N | 96 | None | Heartburn | 8 | Drug |
| 004 | 1 | 18 | F | Y | 93 | None | Heartburn | 5 | Drug |
| 005 | 1 | 16 | F | N | 76 | None | Nausea | 3 | Drug |
| 006 | 1 | 32 | F | Y | 96 | None | Heartburn | 19 | Drug |
| 007 | 2 | 14 | F | Y | 87 | None | None | N/A | N/A |
| 008 | 2 | 14 | F | Y | 94 | None | Sore throat | 1 | Infection |
| 009 | 2 | 12 | M | Y | 96 | None | Headache, mild | 28 | Ibuprofen |
| | | | | | | | cough | | withdrawal |
| 010 | 2 | 28 | F | Y | 100 | None | Bad taste | 28 | Drug |
| 011 | 2 | 19 | F | Y | 93 | None | Rash | 3 | Contact |
| | | | | | | | | | dermatitis |
| 012 | 2 | 44 | F | Y | 92 | None | None | N/A | N/A |
| 013 | 3 | 27 | M | Y | 94 | None | Heartburn | 10 | Drug |
| 014 | 3 | 35 | F | Y | 94 | None | Cold symptoms | 1 | Infection |
| 015 | 3 | 38 | M | Y | 95 | None | Constipation | 2 | Distal |
| | | | | | | | | | intestinal |
| | | | | | | | | | obstruction |
| | | | | | | | | | syndrome |
| 016 | 3 | 23 | M | N | 93 | None | Mild cough, | 10 | Lung |
| | | | | | | | chest pain | | disease |
| 017 | 3 | 31 | M | Y | 100 | None | Weight loss, | 28 | Lung |
| | | | | | | | mild cough | | disease |
| 018 | 3 | 31 | M | Y | 94 | None | Increased | 18 | N/A |
| | | | | | | | sputum | | |

3. Efficacy

In addition to ascertaining the safety of high-dose oral NAC treatment in CF patients, this pilot phase was also designed to provide preliminary assessment of treatment efficacy on numerous outcome measurements, including:

1. Redox balance, as reflected chiefly by (i) whole blood GSH measured by HPLC, and (ii) live blood neutrophil GSH, measured by flow cytometry;

2. Lung inflammation, as reflected chiefly by (i) sputum counts in total live leukocytes and neutrophils (along with % neutrophils in sputum); (iii) plasma/sputum levels of elastase and interleukin-8 (IL-8) measured by spectrophotometry and ELISA (BD Biosciences, San Diego, Calif., USA); and

3. Lung function, as measured by spirometry.

Differences between basal and post-NAC values were studied by matched pair analysis, first, without distinguishing dose cohorts, to detect drug effects, and second, with dose cohorts as a factor, in order to detect potential dose effects. Results show that 4 week-treatment with high-dose oral NAC significantly increased the redox balance and reduced lung inflammation.

In addition, analysis of the CF QOL questionnaire revealed a significant effect on perceived weight gain. With regards to lung function, none of the parameters measured by spirometry showed any change, even as important redox and inflammatory parameters were improved upon treatment. This result was expected, based on the power analysis included in our original proposal. Any sizeable change in lung function likely will require longer treatment and larger group size, which will be implemented in the placebo-controlled phase of the study.

Patients with more severe lung inflammation responded better to drug, notably in terms of the reduction in live sputum leukocytes. In particular, 3 patients (patient 001, patient 011, and patient 016: one in each cohort) were in the normal range of live sputum leukocytes (<0.9 Log₁₀). When these 3 patients were excluded, treatment effects were much more significant (Table 2). In addition, other drug effects became significant, e.g., decreases in sputum IL-8 and % neutrophils.

TABLE 2
Significant drug effects during the phase I trial

| | | Variable | | | | | | | | |
|---|---|---|---|---|---|---|---|---|---|---|
| | | Whole | | Live | Live | Neutrophils | | Elastase | Perceived | |
| | | blood | Neutrophil | sputum | sputum | sputum | IL-8 in | in | weight | |
| Subjects | Statistics | GSH | GSH | leukocytes | neutrophils | (%) | sputum | sputum | gain | FeV1 |
| All | Change | +11% | +17% | −21% | −25% | NS | NS | −44% | Increased | NS |
| (N = 18) | P value | 0.03 | 0.03 | 0.03 | 0.02 | | | 0.02 | 0.01 | |
| 3 | Change | +14% | +25% | −28% | −32% | −9% | −25% | −46% | Increased | NS |
| patients | P value | 0.02 | 0.0003 | 0.005 | 0.003 | 0.04 | 0.02 | 0.02 | 0.05 | |
| excluded | | | | | | | | | | |
| (N = 15) | | | | | | | | | | |

Except for baseline sputum count, drug effect as measured through all the above variables was not dependent on any of the baseline parameters and was not significantly dependent on dose. However, dose cohort 2 (and to a lesser extent cohort 3) showed significant drug effects on additional selected parameters (for example, absolute numbers of neutrophils in blood, which was significantly decreased by 27%), which was more likely related to lower baseline conditions than to dose effect per se. Indeed, cohort 2 was more severely affected with regards to several surrogate markers of disease prior to treatment (lower FEV₁, all infected with P. aeruginosa, and lower perceived weight gain). Thus, cohort 2 may have been more conducive to revealing drug effects than the other two cohorts.

Systemic redox-based therapy is an attractive idea for CF, since redox imbalance is a well-recognized aspect of the disease, yet seldom considered as a bona fide therapeutic target. In that context, the safety and efficacy of high-dose oral NAC has been assessed on redox parameters, inflammation and lung function in CF patients. Having completed the phase 1 trial, it now can be stated that NAC in oral doses as high as about 3.0 g/d do not cause any safety concerns when administered for as long as 4 weeks, thus confirming previous studies in other diseases. The phase 1 trial also brings very strong evidence that high-dose oral NAC can ameliorate significantly both systemic redox stress and lung inflammation in CF, although no effect on lung function was detected after 4 weeks of-treatment.

### Example 2

### Phase II Placebo-Controlled Clinical Trial of High-Dose Oral N-acetylcysteine in CF

Based on the described phase I results, an Investigational New Drug application was submitted to the Food and Drug Administration (IND #73,410), detailing plans for a phase II trial. This application successfully passed the Food and Drug Administration review process. The phase II trial consists of a 12-wk placebo controlled portion, followed by a 12-wk open label portion, both featuring oral NAC treatment at about 2700 mg/day, administered t.i.d. As of June 2006, the 12-wk placebo-controlled portion of this phase II trial was brought to completion.

In compliance with guidelines defined in the Investigational New Drug application, safety data and efficacy data for the primary (sputum cellularity) and main secondary (functional expiratory volume in 1 second) outcome measurements were communicated to the Data and Safety Monitoring Board of the Cystic Fibrosis Foundation before unblinding the study.

1—Enrollment and Compliance

Of the 24 CF study patients who underwent screening, 21 were found eligible for enrollment, based on evidence of ongoing lung inflammation (sputum cellularity >0.9, Log₁₀ scale). Of these 21 patients who received NAC or placebo, 3 were withdrawn for poor compliance before the 12-wk time point. Hence, a total of 18 patients completed the 12-wk time point (% completion=85.7). Among these 18 patients, compliance at the 12-wk time point was excellent, reaching 93.0±1.9% (mean±SE). Compliance was not different between the NAC and placebo groups (93.3±2.3 vs. 92.6=3.2, respectively, N=9 in each group, P=0.9).

2—Safety

The first 12 weeks of this phase II trial (placebo-controlled phase) yielded excellent safety data. No NAC- or placebo-induced serious adverse events were reported. Only 1/18 patients reported adverse events that were likely to be related to treatment (patient in the NAC group), i.e., abdominal discomfort/indigestion which was treated by daily Pepcid AC®. There was no other GI complaint related to NAC (or placebo). Exacerbations of sinus and lung disease affected 5 and 4 out of 18 patients, respectively. The occurrence of exacerbations did not differ between NAC and placebo groups and was not considered to be linked to the trial. Complete blood count and blood chemistry (including liver enzymes) did not show any significant change for either NAC or placebo groups. Hence, safety of high-dose oral NAC administration over the course of 12 weeks showed even better safety results than the 4-week-treatment tested in phase I.

3—Efficacy Data on Lung Inflammation

In this placebo-controlled phase II, results obtained in phase I were confirmed with regard to the ability of NAC to decrease sputum cellularity significantly (Table 3). There was no significant change in sputum cellularity in the placebo group. The significance of this positive effect of NAC on sputum cellularity was further increased when the 6 patients (3 in each group) with confounding treatments administered during the 12-week trial period (prednisone and tobramycin) were excluded from the analysis (Table 3). With these 6 patients excluded, the difference between NAC and placebo groups was statistically significant upon between-group analysis. Hence, the primary outcome measurement in this phase II trial yielded positive results.

4—Efficacy Data on Lung Function

CF lung disease is characterized by the progressive decline in functional expiratory volume in 1 second (FEV1% predicted). The term “FEV1%” as used herein refers to Forced Expiratory Volume during the first second/FVC, where FVC refers to Forced (ExpiratoryVital Capacity (Liters), meaning the maximum volume of air exhaled as rapidly, forcefully and completely as possible from the point of maximum inhalation. Slowing down, stopping or reversing this decline reflect positive effects of a treatment, which generally requires long-term administration. When the 6 patients (3 in each group) with confounding treatments administered during the 12-week trial period (prednisone and tobramycin) were excluded, the NAC group, but not the placebo group, showed a significant increase in FEV₁ % predicted (Table 3). This effect, however, did not reach significance in the between-group analysis, underlining the necessity for larger patient cohorts to ascertain the potential positive effect of oral NAC treatment on CF lung function.

TABLE 3
Chosen drug effects (post-treatment vs. baseline) during the phase II trial

| | | | Placebo: | Between- |
|---|---|---|---|---|
| Subjects | Outcome | NAC: Median | Median | group |
| (N) | measurement | [interquartile] | [interquartile] | analysis |
| All | Sputum | −0.22 | −0.16 | P = 0.825 |
| | cellularity | [−0.34; +0.01] | [−0.51; +0.35] | |
| | (Log10) | P = 0.030 | P = 0.221 | |
| 6 | [Decrease is a | −0.27 | +0.06 | P = 0.025 |
| ex- | positive effect] | [−0.43; −0.19] | [−0.19; +0.77] | |
| cluded | | P = 0.002 | P = 0.218 | |
| All | FEV1 | +1.0 | +2.0 | P = 0.791 |
| | (% predicted) | [−1.0; +6.0] | [−12.0; +10.5] | |
| | [Increase is a | P = 0.150 | P = 0.470 | |
| 6 | positive effect] | +3.5 | −3.0 | P = 0.328 |
| ex- | | [−0.3; +8.5] | [−11.0; +7.0] | |
| cluded | | P = 0.037 | P = 0.328 | |

While the present invention has been described with reference to the specific embodiments thereof, it should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a lung inflammation condition in cystic fibrosis patients, the method comprising the step of:
(a) administering to a patient in need thereof a pharmaceutical composition comprising
(1) an inflammation-reducing amount of N-acetylcysteine, a pharmaceutically acceptable salt of N-acetylcysteine, or a pharmaceutically acceptable derivative of N-acetylcysteine, and
(2) a pharmaceutically acceptable carrier,
thereby modulating the lung inflammation condition.

2. The method according to claim 1, wherein the lung inflammation condition is acute or chronic.

3. The method according to claim 1, wherein in step (a) of the method the pharmaceutical composition is administered systemically by a route selected from the group consisting of orally, buccally, topically, by inhalation, by insufflation, parenterally and rectally.

4. The method according to claim 1, wherein in step (a) of the method, the pharmaceutical composition is administered orally.

5. The method according to claim 4, wherein the inflammation-reducing amount of N-acetylcysteine, a pharmaceutically acceptable salt of N-acetylcysteine, or a pharmaceutically acceptable derivative of N-acetylcysteine in the pharmaceutical composition administered orally is about 1.8 grams per day to about 6 grams per day, and less than or equal to 70 mg per kg per day.

6. The method according to claim 4, wherein the inflammation-reducing amount of N-acetylcysteine, a pharmaceutically acceptable salt of N-acetylcysteine, or a pharmaceutically acceptable derivative of N-acetylcysteine in the pharmaceutical composition administered orally is at least about 1800 mg per day and less than or equal to 70 mg per kg per day.

7. The method according to claim 4, wherein the inflammation-reducing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 70 mg per kg per day.

8. The method according to claim 4, wherein the inflammation-reducing amount of N-acetylcysteine, a pharmaceutically acceptable salt of N-acetylcysteine, or a pharmaceutically acceptable derivative of N-acetylcysteine in the pharmaceutical composition administered orally is at least about 3000 mg per day and less than or equal to 70 mg per kg per day.

9. The method according to claim 1, wherein in step (a) of the method, the pharmaceutical composition is administered parenterally.

10. The method according to claim 9, wherein the inflammation-reducing amount of N-acetylcysteine, a pharmaceutically acceptable salt of N-acetylcysteine, or a pharmaceutically acceptable derivative of N-acetylcysteine in the pharmaceutical composition administered parenterally is about 200 mg NAC to about 20000 mg NAC per dosage unit.

11. The method according to claim 1, wherein the method further comprises the step of
(b) administering a pharmaceutically effective amount of a cystic fibrosis therapeutic agent.

12. The method according to claim 11, wherein the cystic fibrosis therapeutic agent is at least one agent selected from the group consisting of an anti-infective agent, a bronchodilating agent, and an anti-inflammatory agent.

13. The method according to claim 1, the method further comprising the step of
(b) administering a respiratory therapy to the patient.

14. The method according to claim 1, the method further comprising the step of
(b) administering a rehabilitation therapy to the patient.

15. The method according to claim 1, the method further comprising the step of
(b) monitoring lung function of the patient.

16. The method according to claim 1, the method further comprising the step of:
(b) monitoring the lung inflammation by a method comprising the steps of:
(i) collecting a sample of blood or sputum from the patient;
(ii) determining a measure of inflammatory activity in the blood or sputum collected from the patient.

17. The method according to claim 16, wherein the measure of inflammatory activity in the sample of blood in step (ii) is at least one measure selected from the group consisting of a plasma level of neutrophil elastase activity and a plasma level of interleukin-8 activity.

18. The method according to claim 16, wherein the measure of inflammatory activity in the sample of sputum in step (ii) is at least one measure selected from the group consisting of a count of live leukocytes, a count of live neutrophils, a ratio of neutrophils to total leukocytes; a sputum level of neutrophil elastase activity and a sputum level of interleukin-8 activity.

19. A method of treating a redox imbalance condition in cystic fibrosis patients, the method comprising the step of
(a) administering to a patient in need thereof a pharmaceutical composition comprising
(i) a redox-balancing amount of N-acetylcysteine, a pharmaceutically acceptable salt of N-acetylcysteine, or a pharmaceutically acceptable derivative of N-acetylcysteine, and
(ii) a pharmaceutically acceptable carrier, thereby modulating the redox imbalance condition.

20. The method according to claim 19, wherein in step (a) of the method, the composition is administered systemically by a route selected from the group consisting of orally, buccally, parenterally, topically, by inhalation, by insufflation, and rectally.

21. The method according to claim 19, wherein in step (a) of the method the pharmaceutical composition is administered orally.

22. The method according to claim 21, wherein the redox-balancing amount of N-acetylcysteine, a pharmaceutically acceptable salt of N-acetylcysteine, or a pharmaceutically acceptable derivative of N-acetylcysteine in the pharmaceutical composition administered orally is about 1.8 grams per day to about 6 grams per day and less than or equal to 70 mg per kg per day.

23. The method according to claim 21, wherein the redox-balancing amount of N-acetylcysteine, a pharmaceutically acceptable salt of N-acetylcysteine, or a pharmaceutically acceptable derivative of N-acetylcysteine in the pharmaceutical composition administered orally is at least about 1800 mg per day and less than or equal to 70 mg per kg per day.

24. The method according to claim 21, wherein the redox-balancing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 70 mg per kg per day.

25. The method according to claim 21, wherein the redox-balancing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 70 mg per kg per day.

26. The method according to claim 19, wherein in step (a) of the method, the pharmaceutical composition is administered parenterally.

27. The method according to claim 26, wherein the redox-balancing amount of N-acetylcysteine, a pharmaceutically acceptable salt of N-acetylcysteine, or a pharmaceutically acceptable derivative of N-acetylcysteine in the pharmaceutical composition administered parenterally is about 200 mg NAC to about 20000 mg NAC per dosage unit.

28. The method according to claim 19, the method further comprising the step of
(b) administering a pharmaceutically effective amount of a cystic fibrosis therapeutic agent.

29. The method according to claim 28, wherein the cystic fibrosis therapeutic agent is at least one agent selected from the group consisting of an anti-infective agent, a bronchodilating agent, and an anti-inflammatory agent.

30. The method according to claim 19, the method further comprising the step of
(b) administering a respiration therapy to the patient.

31. The method according to claim 19, the method further comprising the step of
(b) administering a rehabilitative therapy to the patient.

32. The method according to claim 19, the method further comprising the step of
(b) monitoring lung function of the patient.

33. The method according to claim 19, the method further comprising the step of:
(b) monitoring the redox imbalance in cystic fibrosis patients by a method comprising the steps of:
(i) collecting a sample of blood or sputum from the patient; and
(ii) determining a measure of redox balance in the sample of blood or sputum.

34. The method according to claim 33, wherein the measure of redox balance in the sample of blood in step (ii) is at least one measure selected from the group consisting of a level of reduced glutathione in whole blood and a level of reduced glutathione in live blood neutrophils.

35. A pharmaceutical kit for treating a lung inflammation condition in cystic fibrosis patients, the kit comprising
a) a first container containing a pharmaceutically effective amount of a cystic fibrosis therapeutic agent, and
b) a second container containing a pharmaceutical composition comprising
(i) an inflammation-reducing amount of N-acetylcysteine, a pharmaceutically acceptable salt of N-acetylcysteine, or a pharmaceutically acceptable derivative of N-acetylcysteine, and
(ii) a pharmaceutically acceptable carrier.

36. The pharmaceutical kit according to claim 35, wherein the pharmaceutical composition in the second container is administered systemically by a route selected from the group consisting of orally, buccally, parenterally, topically, by inhalation, by insufflation, or rectally.

37. The pharmaceutical kit according to claim 35, wherein the pharmaceutical composition in the second container is administered orally.

38. The pharmaceutical kit according to claim 37, wherein the pharmaceutical composition to be administered orally that is in the second container is in an oral form selected from the forms consisting of a tablet, a troche, a lozenge, an aqueous suspension, an oily suspension, a dispersible powder, a dispersible granule, an emulsion, a hard capsule, a soft capsule, a syrup, and an elixir.

39. The pharmaceutical kit according to claim 37 wherein the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is about 1.8 grams per day to about 6 grams per day, and less than or equal to 70 mg per kg per day.

40. The pharmaceutical kit according to claim 37, wherein the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 1800 mg per day and less than or equal to 70 mg per kg per day.

41. The pharmaceutical kit according to claim 37, wherein the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 2400 mg per day and less than or equal to 70 mg per kg per day.

42. The pharmaceutical kit according to claim 37, wherein the inflammation-reducing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 3000 mg per day and less than or equal to 70 mg per kg per day.

43. The pharmaceutical kit according to claim 35, wherein the pharmaceutical composition in the second container is administered parenterally.

44. The method according to claim 43, wherein the inflammation-reducing amount of N-acetylcysteine, a pharmaceutically acceptable salt of N-acetylcysteine, or a pharmaceutically acceptable derivative of N-acetylcysteine in the pharmaceutical composition to be administered parenterally that is in the second container is about 200 mg NAC to about 20000 mg NAC per dosage unit.

45. The pharmaceutical kit according to claim 35, wherein the cystic fibrosis therapeutic agent in the first container is at least one agent selected from the group consisting of an anti-infective agent, a bronchodilating agent, and an anti-inflammatory agent.

46. A pharmaceutical kit for treating a redox imbalance condition in cystic fibrosis patients, the kit comprising
(a) a first container containing a pharmaceutically effective amount of a cystic fibrosis therapeutic agent, and
(b) a second container containing a pharmaceutical composition comprising
(i) a redox-balancing amount of N-acetylcysteine, a pharmaceutically acceptable salt of N-acetylcysteine, or a pharmaceutically acceptable derivative of N-acetylcysteine, and
(ii) a pharmaceutically acceptable carrier.

47. The pharmaceutical kit according to claim 46, wherein the pharmaceutical composition in the second container is administered systemically by a route selected from the group consisting of orally, buccally, parenterally, topically, by inhalation, by insufflation, or rectally.

48. The pharmaceutical kit according to claim 46, wherein the pharmaceutical composition in the second container is administered orally.

49. The pharmaceutical kit according to claim 48, wherein the pharmaceutical composition to be administered orally that is in the second container is in a form selected from the forms consisting of a tablet, a troche, a lozenge, an aqueous suspension, an oily suspension, a dispersible powder, a dispersible granule, an emulsion, a hard capsule, a soft capsule, a syrup, and an elixir.

50. The pharmaceutical kit according to claim 48, wherein the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is about 1.8 grams per day to about 6 grams per day and less than or equal to 70 mg per kg per day.

51. The pharmaceutical kit according to claim 48, wherein the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 1800 mg per day and less than or equal to 70 mg per kg per day.

52. The pharmaceutical kit according to claim 48, wherein the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 2400 mg per day and less than or equal to 70 mg per kg per day.

53. The pharmaceutical kit according to claim 48, wherein the redox-balancing amount of N-acetylcysteine, a pharmaceutically acceptable salt of N-acetylcysteine, or a pharmaceutically acceptable derivative of N-acetylcysteine in the pharmaceutical composition to be administered orally that is in the second container is at least about 3000 mg per day and less than or equal to 70 mg per kg per day.

54. The method according to claim 46, wherein the pharmaceutical composition in the second container is administered parenterally.

55. The method according to claim 54, wherein the redox-balancing amount of N-acetylcysteine, a pharmaceutically acceptable salt of N-acetylcysteine, or a pharmaceutically acceptable derivative of N-acetylcysteine in the pharmaceutical composition to be administered parenterally that is in the second container is about 200 mg NAC to about 20000 mg NAC per dosage unit.

56. The pharmaceutical kit according to claim 46, wherein the cystic fibrosis therapeutic agent in the first container is at least one agent selected from the group consisting of an anti-infective agent, a bronchodilating agent, and an anti-inflammatory agent.
